(19) United States
(12) Patent Application Publication
(10) Pub. No.: US 2020/0330016 A1
(43) Pub. Date: Oct. 22, 2020

(54) WEARABLE OR INSERTABLE DEVICES WITH MICRONEEDLES THAT INCLUDE MECHANICALLY-RESPONSIVE MATERIAL

(71) Applicant: KONINKLIJKE PHILIPS N.V., EINDHOVEN (NL)

(72) Inventors: Javier ESPINA PEREZ, Eindhoven (NL); Pippinus Maarten Robertus WORTELBOER, Eindhoven (NL); Lutz Christian GERHARDT, Eindhoven (NL); Ron Martinus Laurentius VAN LIESHOUT, Geldrop (NL); Mark Thomas JOHNSON, Arendonk (NL)

(21) Appl. No.: 16/772,855

(22) PCT Filed: Dec. 13, 2018
(86) PCT No.: PCT/EP2018/084686

§ 371 Date: Jun. 15, 2020

Related U.S. Application Data
(60) Provisional application No. 62/599,323, filed on Dec. 15, 2017.

(51) Int. Cl. A61B 5/15; A61B 5/145; A61B 5/155; A61B 5/00; A61B 5/157

(52) U.S. Cl. CPC A61B 5/150984; A61B 5/150022; A61B 5/14514; A61B 5/150076; A61B 5/150083; A61B 5/150091; A61B 5/150946; A61B 5/150389; A61B 5/150511; A61B 5/150969; A61B 5/155; A61B 5/6802; A61B 5/157; A61B 5/150099

(57) ABSTRACT

The present disclosure is directed to wearable or insertable devices that allow for ongoing sampling and analysis of biomarkers and self-cleaning. In various embodiments, an apparatus may include a base (102) defining at least one reservoir (104), and at least one microneedle (106, 306, 406, 506, 606, 706, 806, 906) extending from the base. The at least one microneedle may define an inner lumen (409, 509, 609, 709, 809, 909) that fluidly couples the at least one reservoir with tissue of the patient. A mechanically responsive material (670, 770, 870) on an inner surface of the at least one microneedle defining the inner lumen may be reactive to various stimuli to undergo various mechanical responses, such as one mechanical response that purges fluid from the inner lumen of the at least one microneedle and another mechanical response that draws fluid into the inner lumen of the at least one microneedle.

### TECHNICAL FIELD

The present disclosure is directed generally to the use of a wearable or insertable device for the measurement of biomarkers and/or administration of medicine. More particularly, but not exclusively, the various apparatuses, methods, and systems disclosed herein relate to microneedles with mechanically-responsive material that is reactive to stimuli to purge fluid from, or draw fluid into, microneedles.

### BACKGROUND

Ultrafiltration is a commonly used clinical technique where large molecules responsible for poor sensor performance are excluded from a sample matrix. Conventional ultrafiltration is typically accomplished through the use of commercial filter membranes. These filter membranes are often similar to those filters used for hemodialysis and hemofiltration and those that are used ex vivo. Commercially available filter membranes are designed for short-term hemodialysis, hemo-filtration, and/or ultra-filtration, and these commercially available filters have a relatively heterogeneous porous structure. For example a wide variety of membranes (e.g. polysulfone, polyacrylonitrile, polymethacrylates and poly(ethylene) glycol co(polymers), polyamide, cellulose, teflon membranes, and polymer fibres that are spun or weaved into an interconnecting mat-like structures) have been developed to facilitate a rapid rate of water flow and the passage of small and large molecules for short-term hemodialysis, hemo-filtration, and ultra-filtration. These membranes may perform well for short periods of time, but may develop an obstructive pathway due to adhesion of proteins, cells, platelets and thrombi formation, making these membranes undesirable for long-term monitoring of targeted biomarkers.

Generally, biomarkers are substances, structures, or products of processes that can be measured in the body and influence, diagnose, or predict the incidence of outcome or disease. Biomarkers may be categorized into various different categories: 1) screening biomarkers—those that identify the risk of developing a disease; 2) diagnostic biomarkers—those that identify (or rule out) a disease; 3) prognostic biomarkers—those that predict disease progression; 4) pharmacodynamics biomarkers—those that examine pharmacological response; 5) biomarkers that monitor disease activity and clinical response to an intervention; and 6) severity biomarkers—which may act as a surrogate endpoint in clinical trials. Some non-limiting examples of biomarkers include cytokines and interleukins, electrolytes, ketones, triglycerides, insulin, glucose, cholesterol, cortisol, vitamins, anti-oxidants, reactive oxygen species, markers for cancer and anti-cancer therapy, circulating tumor cells, markers of specific medications, micro-ribonucleic acid (miRNA), and the like. Long-term monitoring of biomarkers may be particularly relevant for diagnostic or prognostic biomarkers (e.g. long-term monitoring of insulin levels in diabetic patients).

Implantable porous catheters have been proposed for long-term monitoring and may overcome some of the problems associated with traditional filter membranes. For example, these proposals include the use of an implantable micro-pump, thus eliminating the need for a sample collection device (that may clog) entirely. However, the nature of being an implanted device renders these proposed devices as invasive. Wearable devices have increased in use and have become more accepted in both the clinical environment and for home monitoring. Readings from wearable or insertable devices may be monitored and then may be used to adjust one's lifestyle and/or medication. There exists a need in the art for a minimally invasive, on-skin, wearable apparatus and methods for long-term filtration of large molecules from the sample matrix and monitoring of target biomarkers.

Insertables and/or patches (e.g., e-tattoos) used for the detection and analysis of biomarkers need to be designed in such a way that the body fluids to be analyzed are transported appropriately throughout the analysis process. This includes, for instance, the need of drawing the body fluid (e.g. via a microneedle), washing detection surfaces/chambers, and emptying an analysis circuit (e.g. emptying microneedle as preparation for the next analysis phase). Fluid transport inside such devices is typically realized by using micro-pumps inside the devices or by taking advantage of capillary forces. However, the use of micro-pumps inside devices typically brings the disadvantage that, in general, all microneedles are activated at the same time. And although these pumps are not large, there is limited space in wearable (and especially insertable) devices which makes use of a multiplicity of such pumps prohibitive.

### SUMMARY

The present disclosure is directed to inventive methods and apparatuses for a wearable or insertable device that allows for long-term (continuous or periodic) sampling and analysis of biomarkers. Generally, in one aspect a wearable or insertable device is disclosed, where the wearable or insertable device contains: a substrate (or base) that is affixable to tissue of a patient; a re-generable filter, where the re-generable filter includes a sampling unit coupled to the substrate, the sampling unit adapted to obtain one or more fluid samples from the tissue of the patient, and a re-generation unit adapted to apply fluid back-flow to the sampling unit; a module, fluidly coupled with the sampling unit, where the module is adapted to determine a presence or measure of at least one biomarker contained in the one or more fluid samples; and, a power unit operably coupled with the re-generation unit.

In some aspects the sampling unit further comprises a plurality of microneedles, in fluid communication with at least one reservoir, the reservoir adapted to provide a sample to the detection or assay modules. In other aspects, the plurality of microneedles each have an inner diameter of about 1.5 μm to about 2 μm and an inner-lumen with surface chemical gradient coatings, wherein the surface chemical gradient is switched by a signal from the detection module or power unit. In still other aspects, the plurality of microneedles each have an inner-lumen coated in a biocompatible material known for anti-fouling.

In some aspects, the re-generation unit actively applies fluid back-flow to the sampling unit. In other aspects, the re-generation unit further contains a piezo-electric unit adapted to reversibly empty and clean the sampling unit by ultrasound pressure waves generated by the piezo-electrical unit. In still other aspects, the re-generation unit is further arranged to apply a switchable electric field across an insulating layer to an inner lumen of each microneedle of a plurality of microneedles. In still other aspects, the re-generation unit further contains light elements adapted to produce shock waves in fluid back-flow through the sampling unit. In still other aspects, the re-generation unit further contains a rotating element arranged to induce up-flow and back-flow of non-Newtonian body fluid through the sampling unit.

Generally, in another aspect, a method of monitoring a physiological condition of a patient is disclosed, where the method includes: placing a wearable or insertable device on a patient; collecting one or more fluid samples with the wearable or insertable device, where the one or more fluid samples are collected through a sampling unit; preventing clogging of the sampling unit, where the prevention includes introducing fluid back-flow through the sampling unit; determining a measure or presence of at least one biomarker based on the collected one or more fluid samples; and, inferring the physiological condition of the patient based on the determined measure or presence of the at least one biomarker. In some aspects of the method, the sampling unit further contains a plurality of microneedles and the preventing clogging of the sampling unit includes each microneedle having an inner-lumen coated in a biocompatible material known for anti-fouling.

In some aspects of the method, preventing clogging of the sampling unit includes applying a reversed fluid flow through under-pressure initiated by a plurality of ultrasound pressure waves generated by a piezo-electrical unit. In other aspects of the method, preventing clogging of the sampling unit includes applying an electric field across an insulating layer to an inner lumen of each of a plurality of microneedles. In still other aspects of the method, preventing clogging of the sampling unit includes applying an external force to the wearable or insertable device. In still other aspects of the method, preventing clogging of the sampling unit includes switching surface chemistry inside the plurality of microneedles, each of the plurality of microneedles having an inner lumen with gradient coatings and an inner diameter of about 1.5 μm to about 2 μm. In still other aspects of the method, preventing clogging of the sampling unit includes using shock waves to apply fluid back-flow through the sampling unit. In still other aspects of the method, preventing clogging of the sampling unit includes interrupting rotation of a spinning rod inside each of a plurality of microneedles. In still other aspects of the method, the method further includes exchanging data regarding the physiological condition of the patient with one or more remote computing devices.

Generally, in another aspect a method of monitoring a physiological condition of a patient is disclosed, the method including: placing a wearable or insertable device on the patient, where the wearable or insertable device contains a substrate that is affixable to tissue of a patient, a re-generable filter, where the re-generable filter contains a sampling unit coupled to the substrate that is adapted to obtain one or more fluid samples from the tissue of the patient and a re-generation unit adapted to apply fluid back-flow to the sampling unit, a module, fluidly coupled with the sampling unit, where the module is adapted to determine a presence or measure of at least one biomarker contained in the one or more fluid samples, and a power unit operably coupled with the logic or the re-generation unit; collecting one or more fluid samples with the wearable or insertable device, where the fluid sample is collected through a sampling unit; preventing clogging of the sampling unit, where the prevention includes introducing fluid back-flow; determining a measure or presence of at least one biomarker based on the collected one or more fluid samples; and, inferring the physiological condition of the patient based on the determined measure or presence of the at least one biomarker.

In some aspects of the method, preventing clogging of the sampling unit includes each microneedle having an inner-lumen coated in a biocompatible material known for anti-fouling.

In another aspect, a medical device may include: a base defining at least one reservoir; at least one microneedle extending from the base, wherein the at least one microneedle is insertable into tissue and defines an inner lumen that fluidly couples the at least one reservoir with the tissue; and a mechanically responsive material disposed on an inner surface of the at least one microneedle, wherein the inner surface of the at least one microneedle defines the inner lumen of the at least one microneedle, and the mechanically responsive material is reactive to a stimulus to undergo one or more mechanical responses.

In various embodiments, the medical device may further include one or more stimulation components that may be selectively activated to provide the stimulus to the mechanically responsive material. In various embodiments, at least one mechanical response of the one or more mechanical responses of the mechanically responsive material purges fluid from the inner lumen of the at least one microneedle. In various embodiments, the medical device may further include a valve positioned between the mechanically responsive material and the at least one reservoir. In various embodiments, the valve may be closable such that the at least one mechanical response of the mechanically responsive material purges fluid from the inner lumen into the tissue. In various embodiments, the valve may be openable such that the at least one mechanical response of the mechanically responsive material purges fluid from the inner lumen into the at least one reservoir.

In various embodiments, at least one of the one or more mechanical responses of the mechanically responsive material draws fluid into the inner lumen of the at least one microneedle. In various embodiments, a first mechanical response of the one or more mechanical responses may include expansion of the mechanically-responsive material and a second mechanical response of the one or more mechanical responses may include contraction of the mechanically-responsive material.

In various embodiments, the mechanically responsive material may be divided into a plurality of individually-reactive segments that are arranged along a length of the at least one microneedle, wherein stimulation of the plurality of individually-reactive segments in a predetermined sequence may cause the individually-reactive segments to expand in accordance with the predetermined sequence to purge fluid from, or draw fluid into, the inner lumen.

In various embodiments, the mechanically-responsive material may include one or more paddles that extend from the inner surface into the inner lumen, wherein the one or more paddles are operable to purge fluid from, or draw fluid into, the inner lumen. In various embodiments, the one or more paddles may include a plurality of individually-operably paddles that are operably in a predetermined sequence to purge fluid from, or draw fluid into, the inner lumen. In various embodiments, one or more of the paddles may be operable as a valve to selectively open and close the inner lumen. In various embodiments, at least one given paddle of the one or more paddles may include a folding actuator that is operable to fold the given paddle upon itself.

In various embodiments, the mechanically-responsive material may be transitionable between a hydrophilic state in which the mechanically-responsive material attracts fluid, and a hydrophobic state in which the mechanically-responsive material repels fluid. In various embodiments, the mechanically-responsive material is constructed with electroactive polymer (“EAP”) or magnetorheological elastomer (“MRE”). In various embodiments, the mechanically-responsive material may be constructed with shape-memory polymer or with light-activated liquid crystal networks.

In various embodiments, the stimulus may include heat, electricity, electromagnetic radiation (i.e. visible or invisible light), one or more acoustic waves, a magnetic field, or any combination thereof.

Where used herein the term “affixed” or “affixable” may include the removable attachment of a device to tissue, for example with an adhesive material to the outer surface of skin. Additionally, or alternatively, the term “affixed” or “affixable” may also include the insertion and placement of a device into internal tissue.

### BRIEF DESCRIPTION OF THE DRAWINGS

In the drawings, like reference characters generally, but not exclusively, refer to the same parts throughout the different views. Also, the drawings are not necessarily to scale, emphasis instead generally being placed upon illustrating the principles of the disclosure.

FIG. 1 illustrates a cross-section of human skin with an embodiment of a wearable device.

FIG. 2 depicts an example method for determining a physiological condition of a patient.

FIG. 3 depicts an embodiment of an apparatus configured with selected aspects of the present disclosure that is inserted into tissue of a patient.

FIGS. 4A, 4B and 4C depict one example of how a microneedle may be cleared of obstructions and/or adhesions.

FIG. 5 depicts an example of a microneedle with electrowetting elements.

FIGS. 6A, 6B and 6C depict one embodiment of a microneedle that includes mechanically-responsive material.

FIGS. 7A and 7B depict another embodiment of a microneedle that includes mechanically-responsive material.

FIGS. 8A and 8B depict another embodiment of a microneedle that includes mechanically-responsive material.

FIGS. 9A, 9B and 9C depict another embodiment of a microneedle that includes mechanically-responsive material.

FIG. 10 depicts another example method for determining a physiological condition of a patient.

### DETAILED DESCRIPTION

A challenge in taking blood samples (either periodic or continuous) by a wearable or insertable device is separating various component cells from plasma proteins and other molecular biomarkers of interest. This may be challenging due to the adhesion of proteins, cells, platelets, etc. that may create an obstruction in the sampling pores or filter; thus, it is desirable to prevent this clogging. It may also be desirable to separate blood cells, platelets, and target biomarkers (e.g. plasma proteins, small molecules like cholesterol and glucose). By filtering out various molecules and preventing clogging of the sampling pore, accurate long-term (either periodic or continuous) readings of biomarkers in order to track health of an individual patient may be achieved through the use of wearable or insertable devices.

A wearable or insertable device described herein may include a re-generable filter, an assay module for performing a biochemical test, a detection module for detecting the presence of targeted biomolecules, a user interface, a power unit, and/or a logic. In various embodiments, the re-generable filter may also include a sampling unit for the collection of samples and a re-generation unit that prevents long term obstruction of the filter. The sampling unit may be configured to collect samples from the patient, and may further include pores of defined sizes, charged surfaces, microneedles of a particular size to filter out undesirable molecules, etc. While examples described herein refer to the use of “microneedles”, this is not intended to be limiting. For example, electrospun fibers may also be used in order to filter out undesired molecules, and the apparatuses and methods described herein may also be used in conjunction with electrospun fibers or other filtering mechanisms known in the art.

In some embodiments the sampling unit is comprised of an array of microneedles capable of reaching anatomical structures such as small blood vessels and/or capillaries or interstitial fluid. In some embodiments, the inner diameter of the microneedles may be large enough to accommodate the passage of blood plasma, but small enough to prevent the passage of red blood cells (RBCs), white blood cells (WBCs), and platelets into the microneedle. There are a variety of types of WBCs, for example neutrophils, basophils, eosinophils, lymphocytes, monocytes, macrophages, etc., and as such there is a wide range in the size of WBCs. Typically, the diameter of WBCs range from about 6.8 μm to about 30 μm. RBCs are typically disc shaped, and have diameters that range from about 6.2 μm to about 8.2 μm and thicknesses of about 2 μm to about 2.5 μm. Platelets typically range from about 2 μm to about 3 μm. Therefore, a microneedle with an inner diameter of about 1.5 μm to about 2 μm may prevent the passage of these types of cells into the microneedle, and thus into the wearable or insertable device.

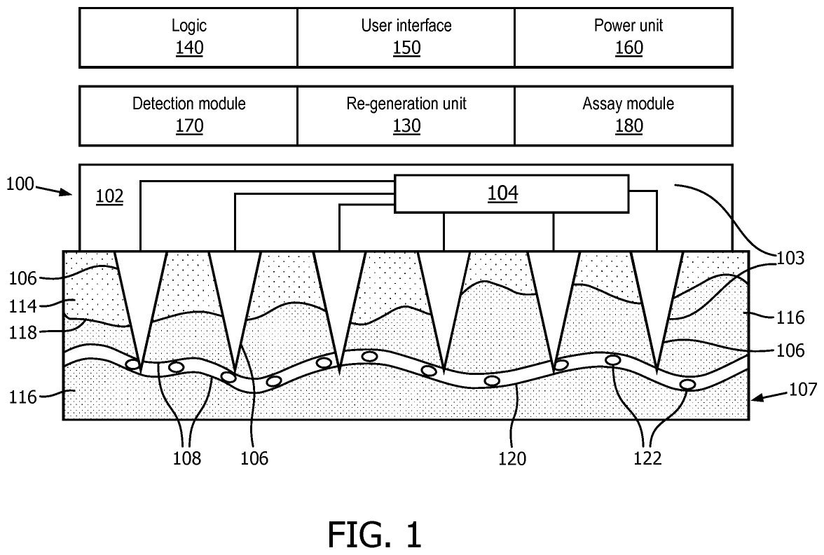

Referring to FIG. 1 (which is not drawn to scale), an embodiment of a wearable device 100 disclosed herein is illustrated. In the illustrated embodiment, the wearable device 100 is in the form of a thin patch or tattoo-like structure with a user interface 150, a power unit 160, and logic 140. In various embodiments, the wearable device 100 is affixed to a patient by means of a substrate 102 (which may be flexible or rigid depending on the application). For example, microneedles 106 disposed on one side of substrate 102 (e.g., a bottom side in FIG. 1) may be inserted (e.g., pierced) through tissue 107, which in some cases may be the surface of the patient's skin. Assuming that is the case, tissue 107 may include an epidermis 114 separated from a dermis 116 by an epidermal-dermal junction (“EDJ”) 118. The tips 108 of microneedles 106 may reach one or more capillaries 120 (which may carry arterial or venous blood). Assuming the biomarkers 122 sought to be assayed are contained in the capillaries 120, a fluid sample may be collected via one or more microneedles 106, such that the RBCs, WBCs, and platelets are not collected due to the size constraints of the inner diameter of the microneedle. Although described primarily in terms of blood samples, that is not to be understood as limiting, in alternate embodiments the biomarkers sought to be analyzed may be in, and samples may be collected from other sample types including, but not limited to saliva, sweat, lymph fluid, urine, interstitial fluid, feces, exhaled breath concentrated, and the like. In these and other embodiments, the size of the inner diameter of the microneedle may vary based on the intended use and targeted biomarker. For example, the inner diameter of the microneedle may be larger than 1.5 μm to about 2 μm where the targeted biomarker is larger than these constraints.

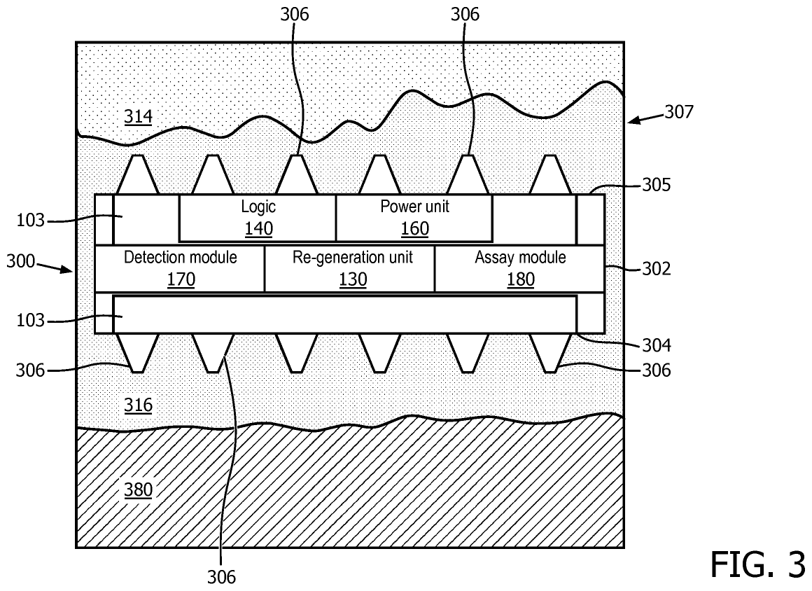

The wearable device 100 of FIG. 1 also includes a re-generable filter. The re-generable filter may include a sampling unit 103 and a re-generation unit 130. The sampling unit 103 may include of a collection of components, such as microneedles 106 described previously and, in some embodiments, at least one reservoir 104 for storing the collected sample (though not necessarily all together from the individual microneedles). In other embodiments, the device may be inserted beneath the tissue surface, as is described below with respect to FIG. 3.

Over time, the components of the sampling unit 103, for example microneedles 106, may become obstructed due to the aggregation and/or adhesion of proteins, cells, platelets, etc. With conventional approaches the pores of sampling units 103 (e.g. the inner lumens of the microneedles) may clog within hours of continuous or periodic sampling. Accordingly, in various embodiments, the inner-lumen of the microneedles 106 may be coated with a biocompatible coating known to enhance anti-fouling, for example albumin or poly(ethylene)glycol based coatings. These biocompatible coatings may slow the obstruction of the openings of the microneedles by minimizing adhesion of proteins, cells, etc. to the inner lumen of the microneedle. However, in some instances these coatings may be not sufficient to prevent obstructions during long-term use. Other methods of avoiding obstructing the microneedles 106 of the sampling unit 103 include, but are not limited to, rinsing or purging the microneedles with an anticoagulant, for example heparin, a coating on the inner lumen of microneedle that entraps air in order to prevent the clogging of the tip of the microneedle, and/or the use of actuation or vibration to prevent and break up obstructions. This rinsing or purging of the microneedles may be driven by various techniques, including, but not limited to, the use of an electric field (e.g. electrowetting, the use of surface gradients, etc.).

Obstructions may develop in the sampling unit 103 despite use of conventional methods of prevention. This may be especially true in long-term monitoring, where there may be, as time progresses, a time dependent deterioration of the ability of the sampling unit 103 to effectively collect a sample. Thus, the ability of the wearable device 100 to be used for long-term monitoring depends, in part, on the ability to prevent and/or clear these obstructions. The re-generation unit 130 may prevent long-term obstruction of the sampling unit 103 by introducing the back-flow of fluid through the sampling unit 103 which may dislodge and force out any proteins, cells, etc. that have adhered to the inner lumen(s) of the microneedle(s). In some embodiments, this fluid may be fluid that was able to pass through the sampling unit 103 (e.g., the microneedles 106) and may have already been analyzed by the wearable device 100. Alternatively, or additionally, the fluid may be recently collected and sourced from a small reservoir (e.g., 104). Such a reservoir, where present, may also contain additional elements (e.g. other chemicals for aiding in combatting obstructions, such as anti-coagulants). Additionally, the back-flow of fluid may create conditions that are unfavorable for the formation of these adhesions and obstructions in the sampling unit 103. Various mechanisms for generating and applying back-flow and/or under-pressure by the re-generation unit 130 are described herein.

In some embodiments, the re-generation unit 130 may include a piezo-electric unit that may use electricity to generate pressure to actively re-generate the filter (e.g., the sampling unit 103, which as noted above includes the microneedles 106), including inner-lumen(s) of the microneedle(s) 106, by applying reverse fluid flow. In some embodiments the piezo-electric unit may include one or more vibrating piezo crystals and/or one or more capacitive micromachined ultrasonic transducers (CMUT) affixed to or positioned within a close proximity to the microneedles. The piezo-electric unit may produce needle wall vibrations and vacuum bubbles within the fluid contained within the inner-lumen of the microneedle, including the target analyte(s). These bubbles may grow, oscillate, and collapse/implode with enough intensity to clear the inner-lumen from adsorbed or adhering biomolecules. In other words, the ultrasound waves produced by the piezo-electric unit may create short, intense fluid flows through cavitation techniques, which act to dislodge and force out any proteins, cells, etc. that may have adhered to the inner lumen of the microneedle.

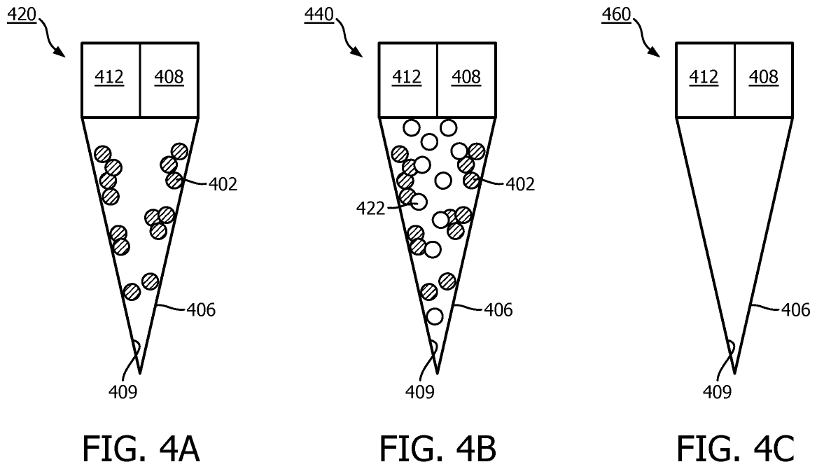

In some embodiments, a continuous flow of fluid into a patient's tissue or collection reservoir may be achieved in additional to and/or simultaneously with re-generating the filter, for example by using a piezo-electric unit. In some embodiments, this continuous fluid flow into a patient's tissue or collection reservoir may be facilitated by use of geometrically tapered microneedles and/or geometrically tapered inner-lumens of microneedles, using coatings and/or other techniques to generate switching between hydrophobic and hydrophilic stated within the inner-lumen of the microneedle, and/or use of electric charges within or near the microneedles, including, but not limited to the use of electrowetting as described herein. In some embodiments the fluid flow may be directed into the device, for example into a reservoir. In other embodiments, the fluid flow may be directed into a patient's tissue. In still other embodiments, the directionality of the fluid flow may be determined by the placement of the piezo-electric unit relative to the microneedle. As an illustrative, non-limiting example, where a piezo-electric crystal(s) is placed at the base of the microneedle (e.g. by the substrate; as illustrated in FIGS. 4A, 4B and 4C) the fluid flow may be directed into a patient's tissue. Alternatively, where a piezo-electric crystal(s) is placed at the microneedle tip (not shown in FIGS. 4A, 4B and 4C) the fluid flow may be directed into the device. Further embodiments may include an accelerometer, which may provide a device information regarding a gravity direction, which may allow a device to identify the most suitable actuation segments for use in filter re-generation.

FIGS. 4A, 4B and 4C illustrate a technique for preventing clogging of the sampling unit, as well as an apparatus that embodies the use of a cavitation technique with a piezo-electric unit for such prevention. FIG. 4A illustrates a stage 420 of a technique of cleaning a microneedle and clearing its inner lumen 409 of obstruction. In stage 420 a microneedle 406, or a plurality thereof, has adhesions/obstructions to be cleaned. The apparatus of FIG. 4A contains at least one microneedle 406 with various blood cells and/or bodily liquids 402 and the like adhered to a surface of an inner lumen 409 of the microneedle 406, a piezo-electric unit 408, and a power input 412. In some embodiments, including that depicted in FIGS. 4A, 4B and 4C, the piezo-electric unit 408 may contain its own power source 412 (e.g. a battery). However in other embodiments, the piezo-electric unit 408 may draw power from the power source 160 of the wearable device 100. In FIG. 4B, which demonstrates a stage 440 of the aforementioned technique, microneedle 406 is actively being cleared of obstruction, e.g., by way of producing bubbles 422 through acoustic cavitation generated by the piezo-electric unit 408 in the form of bubbles 422. Consequently, these bubbles 422 act to dislodge and force out any proteins, cells, etc. (e.g. adhesions 402 of varying compositions) that may have adhered to the inner lumen of the microneedle 404.

FIG. 4C illustrates a final stage 460 of cleaning a microneedle 406 in which it is cleared of obstruction. The collapse/implosion of bubbles 422 produced at stage 440 generates a fluid back-flow which clears the microneedle 406 of any dislodged debris. Although only a single microneedle 406 is illustrated in FIGS. 4A, 4B and 4C, this is not to be understood as limiting as the method and apparatuses illustrated therein may be used with a single microneedle or a plurality of microneedles.

In still other embodiments, the re-generation unit 130 may function by adjusting the capillary forces within the microneedles. For example, adjusting the capillary forces within the microneedles may be achieved through the process of electrowetting, during which an electric field is applied across a layer insulating the inner surface of the microneedle, causing the surface tension to be altered from hydrophilic, where the fluid is drawn to the interior of the microneedle (for example, for use during sample collections) to hydrophobic, where the fluid is repelled from the interior of the needle (for example, for use in releasing the collected sample from the microneedle) However, in some embodiments it may be that the repelling from the inner surface is not immediate. Furthermore, the electric field, which induces the change in the surface tension from hydrophilic to hydrophobic, can be repeatedly applied and removed. This repeated application, and corresponding switching of the surface tension back and forth between hydrophilic and hydrophobic, may flush fluid through the microneedle and clear any adhesions or obstructions present. In some embodiments, the switching of the surface tension back and forth between hydrophilic and hydrophobic, in combination with the fluid flow generated thereby, may be also used for breaking apart obstructing substances and/or adhesions from the interior surfaces of a microneedle.

The surface chemistry of the inner-lumen of the microneedles may also be altered using other techniques. For example, the inner-lumen of the microneedles may be coated such that the coating is a hydrophobic to hydrophilic gradient (or vice versa) from the tip of the microneedle to the opposing end of the microneedle. Such a gradient may induce back-flow through the inner-lumen of the microneedle and may dislodge and force out any proteins, cells, etc. that have adhered to surfaces of the inner lumen of the microneedle. These gradient coatings may be present in the inner-lumen of the microneedle at all times, or they may be selectively applied as desired. For example, the surface chemistry of the inner lumen of the microneedles may be altered through the use of light, such that an interruption in the supply of the target analyte (e.g. biomarker) to the assay and/or detection unit signals a light to cause the surface chemistry to be adjusted to form a gradient.

Although described herein in terms of using electrowetting or gradients, the use of surface chemistry to induce back-flow and thus prevent the formation of obstructions in the microneedles is not so limited. Any method of adjusting capillary forces known in the art capable of alternating surface tension and adhesive forces in order to apply back-flow and induce the dislodge any proteins, cells, etc. that have adhered to the inner lumen of the microneedle may be used.

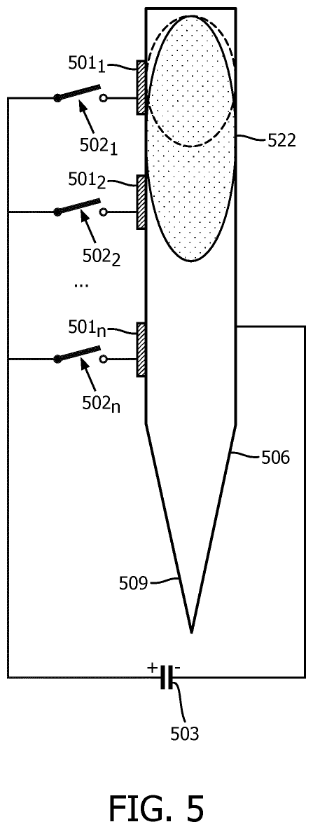

In other embodiments, the re-generation unit 130 may use electrowetting to activate electrode elements and dynamically change the droplets of fluid inside the inner-lumen of the microneedle, as illustrated in FIG. 5. This electrowetting may occur at liquid-liquid or liquid-air interfaces inherent in the inner-lumen of the microneedle. As illustrated in FIG. 5, one or more electrowetting electrodes 501₁-501ₙ may be circumferentially integrated into the microneedle 506 itself, including, but not limited to, integration into the inner-lumen 509 of the microneedle 506. The electrodes, as illustrated in FIG. 5, may be connected to one or more switches (502₁-502ₙ) powered by a battery 503. The sequential activation and multiplexing of the electrode elements 501₁-501ₙ (for example, by the one or more switches) may result in the fluid contained in the inner-lumen 509 of the microneedle 506, including, but not limited to, any target analyte(s) 522 (e.g. biomarker(s)) present, to dynamically change, thus changing the angle of contact between the inner-lumen 509 of the microneedle 506 and the bioanalyte 522. In some embodiments, this may result in the angle of the fluid (bioanalyte/biomarker 522), including any target analyte(s) present in the inner-lumen 509, to be reduced. This sequential activation and multiplexing may induce a gradient and/or a pumping action, which may lead to fluid flow. In some embodiments, fluid may flow from the inner-lumen 509 of the microneedle 506 into tissue of the patient. In other embodiments, fluid may flow from the inner-lumen 509 of the microneedle 506 into a container within the device, such as a reservoir or a waste container (not illustrated in FIG. 5).

In still other embodiments, the re-generation unit 130 utilizes external force to create fluid flow out of the microneedles. External pressure may be applied to a chamber inside the wearable or insertable device 100 which generates fluid flow through and then out of the inner-lumen of the microneedle (i.e. back-flow). This back-flow may dislodge and force out any proteins, cells, etc. that have adhered to the inner lumen of the microneedle, thus removing any obstructions and allowing sampling and monitoring to continue. In some embodiments, the fluid creating the fluid back-flow may be fluid that was able to pass through the filter and may have been previously analyzed by the wearable or insertable device. Alternatively, or additionally, the fluid may be recently collected and sourced from a small reservoir (e.g., 104). Such a reservoir, where present may also contain additional elements (e.g. other chemicals for aiding in combatting obstructions). In some embodiments the external pressure may be from a wearer pressing with, for example, a finger on a designated area of the device. In other embodiments, the external pressure may be from an alternate mechanical source. When the external pressure is removed, both the chamber and the wearable or insertable device may be returned to their original state due to the elasticity of the device and/or chamber. Once returned to the original state, sample collection and monitoring may continue as usual.

In other embodiments, shock waves may be used to generate and apply back-flow and/or under-pressure. Generally, shock waves may propagate through any obstruction present in the sampling unit (e.g., inner-lumens of the microneedles) and this may cause a change in pressure, temperature, density, etc. in the obstruction(s). These changes may cause any obstructions present, such as adhesions of proteins, cells, etc. to be dislodged and forced out of the inner-lumen of the microneedle. Any method of producing shock waves known in the art may be used; however, it may be that light or lased-induced liquid jet production is used. Generally, the process of laser-induced liquid jet production involves inserting an optical fiber into a capillary tube filled with water. A laser beam is then transmitted via the optical fiber and produces water vapor bubbles toward the capillary exit. The water is then expelled from the capillary exit by the expanding bubbles. The collapse and rebound of microbubbles and water flow generated by the emanation of water creates shock waves. With respect to a wearable or insertable device, an optical fiber may be inserted into the inner-lumen of the microneedles as necessary to prevent or clear any obstructions. Alternatively, the optical fiber may remain in place, e.g., within the inner-lumen of the microneedle, and may be activated as necessary. The transmission of a laser beam via the optical fiber in the fluid-filled inner-lumen of the microneedle may create bubbles, which may then dislodge any obstructions or adhesions to the inner-lumen. The bubbles may also cause the fluid and/or any dislodged obstructions or adhesions to be expelled from the inner-lumen of the microneedle.

In other embodiments, the Weissenberg effect may be used to induce back-flow of fluid through the microneedle. The Weissenberg effect is a physical phenomenon where a spinning rod, or other rotating element, is inserted into a non-Newtonian solution of liquid. The liquid, rather than being cast outward by the spinning rod, is drawn towards the rod and rises up around it. In some embodiments, the wearable or insertable device may further contain a spinning rod inside of the microneedle such that the spinning rod and Weissenberg effect aid in the collection of a sample and pulling of fluid through the inner-lumen of the microneedle. The spinning of the rod within the microneedle may be powered by the power unit of the wearable or insertable device. When the rotation of the rod is interrupted, the fluid that was rising up around the rod will flow back (towards, and ultimately through, the tip of the microneedle) without further intervention due to inertia. This back-flow of fluid upon the cessation of the rod spinning dislodges and forces out any obstructions or adhesions of proteins, cells, etc. that may be present attached to or within the inner-lumen of the microneedle.

Again referring to FIG. 1, the illustrated embodiment of the wearable or insertable device 100 further includes a detection module 170 which detects the presence of targeted biomolecules. For example, the detection module 170 may be used to detect the presence of glucose or cholesterol in a sample. Where the desired information is presence/absence data for a target biomolecule this may be the conclusion of the analysis. However, where quantitative measurement may be desired, an assay module 180 may perform a biochemical assay on the sample. The assay module 180 may perform biochemical assays using chemical, electrical, optical, or other energy-based approaches, and/or any other conventional assay technique. In some embodiments, the detection module 170 and assay module 180 may be incorporated into the same physical space and/or into a single module with both functions. In some embodiments, the assay module 180 may use chemical or enzymatic techniques and optical measuring device. For example, a chemical reaction may result in a gradient of color change to indicate a measurement. This color change may then be read and interpreted by an optical reader. In other embodiments, the assay module 180 may be configured to use techniques such as, or similar to, the following: enzyme-linked immunosorbent assay (“ELISA”), which uses antibodies and color change or fluorescence to identify a biomarker; western blotting (or “protein immunoblot”); eastern blotting; Southern blotting; northern blotting; southwestern blotting, electrophoresis, mass spectroscopy, gene or protein arrays, flow cytometry, etc. In other embodiments measurement may include transcriptome assay using e.g. micro-array technique for gene expression studies or quantitative polymerase chain reaction (PCR). In still other embodiments measurement may include epigenetic markers, such as DNA methylation, histone acetylation and miRNA.

The wearable or insertable device 100 may further contain a user interface 150, as illustrated in FIG. 1. The user interface 150 may include data input and/or output components and may also be both attached and integrated directly with the device or may be separated therefrom for ease of use and access. For example, a user may input data through the user interface 150 via a touchscreen incorporated on the wearable or insertable device 100, audio input systems such as voice recognition systems, microphones, etc. In other embodiments, a user may interface with the wearable device 100 utilizing a remote computing device (e.g. computer, smart phone, smart watch, etc.) wirelessly coupled with the wearable or insertable device 100 via the logic 140. For example, in some embodiments, a user may input a selection of the type of biochemical analysis to perform. Data may be output to a user via a visual display, such as a liquid crystal display (LCD) on the wearable device and/or through non-visual outputs such as audio and tactile output. In other embodiments, a user may receive notifications or output information from the wearable device 100 through a secondary device (e.g. computer, smart phone, etc.) wirelessly coupled with the wearable or insertable device 100 via the logic 140. For example, in some embodiments, the user interface 150 may output information to the user indicating the results of a biochemical analysis and/or may indicate that it is desirable for the re-generation unit to activate back-flow to cleanse the sampling unit 103.

The power unit 160 may take various forms, such as one or more batteries, which may or may not be rechargeable, e.g., using one or more integrated solar cells (not depicted) or by periodically being connected to a power source. Furthermore, the power unit 160 may be various power harvesting techniques wherein electrical power is generated from the heat of the wearer of the device, electrochemical harvesting techniques from ions within the human body and/or biological fuel cells, etc. Alternatively, power harvesting may occur as a result of generation of electrical potential from kinetic energy. In still further embodiments, power may be generated from solar or other devices to power the logic and other modules while also charging batteries for later use. Even further embodiments may allow for power to be generated through inductive coupling with an external inductive field source. Of course, in some embodiments, one or more of the power units may be omitted in favor of external power and/or computing resources, such as a computing device that may be operably coupled, for instance, with the logic 140.

The logic 140 may take various forms, such one or more microprocessors that execute instructions stored in memory (not depicted) which may be functionally connected with the logic or other supporting circuitry. Other forms of logic may include a field-programmable gate array (“FPGA”), an application-specific integrated circuit (“ASIC”), or other types of controllers and/or signal processors. In various embodiments, the logic 140 may control various aspects of operation of apparatus 100 described herein. In some embodiments, the logic 140 may include one or more wired or wireless communication interfaces (not depicted) that may be used to exchange data with one or more remote computing devices using various technologies, such as Bluetooth, Wi-Fi, USB, etc. In various embodiments, the logic 140 may be operably coupled with one or more re-generation units 130, e.g., via one or more busses (not depicted), and may be configured to operate one or more re-generation units 130 to induce back-flow of fluid through the sampling unit.

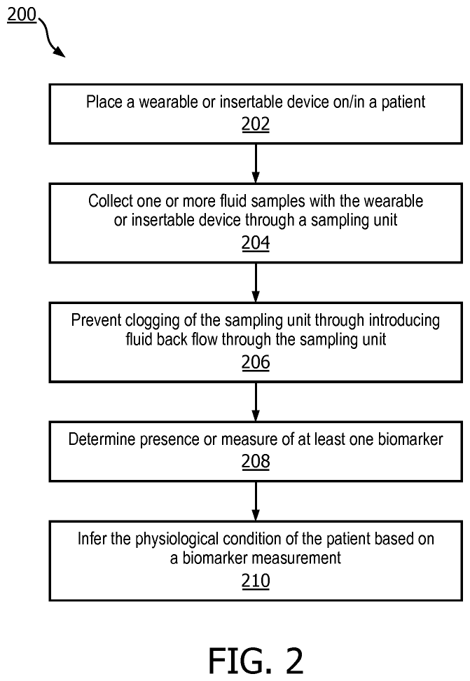

Referring now to FIG. 2, an example method 200 for determining a physiological condition of a patient that may be practiced, for instance, using the apparatus (100) described herein is depicted. While operations of method 200 are depicted in a particular order, this is not meant to be limiting. In various embodiments, one or more operations may be added, omitted, and/or reordered.

At block 202, a wearable or insertable device configured with selected aspects of the present disclosure may be placed onto, or inserted into, tissue of a patient, such as the patient's skin. In some embodiments, this may include inserting at least one microneedle into the tissue. The wearable device may be adhered to the patient's tissue in various ways. In some embodiments in which multiple microneedles are employed, insertion of the microneedles into the tissue may itself affix the wearable device to the patient's tissue. In other embodiments, the microneedles may remain in a recessed position and are deployed or launched at a later time point after insertion into the tissue. Additionally or alternatively, various biocompatible adhesives may be applied to the wearable or insertable device to affix the wearable device to the patient's tissue. In some embodiments, an adhesive bandage or other suitable component may be used to “tape” the wearable or insertable device to the patient's tissue. In other embodiments, the device may be inserted beneath the tissue surface, as is described below with respect to FIG. 3. In some embodiments, the adhesive may serve multiple purposes. For example, in some embodiments the adhesive may also be used to seal blood vessel following surgical procedures and the like (e.g. fibring glue, cyanoacrylate, electrocuring glue, etc.). In other embodiments, the adhesive may be a gel patch or a silicone rubber patch for use in coupling acoustic (ultrasounds) waves generated by a piezo-electric unit to patient tissue.

At block 204, the wearable or insertable device collects one or more fluid samples through a sampling unit. In some embodiments the sampling unit contains microneedles with an inner diameter of about 1.5 μm to about 2 μm, so as to filter out RBCs, WBCs, and platelets from fluid passing through the microneedle(s), and thus into the wearable or insertable device. In some embodiments the collection of fluid samples may be continuous for a defined time period or until a fixed activity is complete. In other embodiments, the samples are collected at various time points. In some embodiments, the period of time in which samples are collected may be defined by the user, third-party, necessity of the biomarker being monitored, etc. In other embodiments, the period of time in which samples are collected may remain indefinite.

At block 206, the wearable or insertable device uses fluid back-flow through the sampling unit to prevent the clogging of the sampling unit and filter. In other words, the re-generation unit prevents long-term obstruction of the sampling unit (e.g. microneedles) by introducing fluid back flow into the sampling unit which may dislodge and force out any proteins, cells, etc. that have adhered to the inner lumen of the microneedle. As described above, there are multiple embodiments for generating fluid back-flow by the regeneration unit, these include, but are not limited to: the regeneration unit further comprising a piezo-electric unit; adjusting the capillary force/surface chemistry through electrowetting and/or light; applying external pressure; using shock waves; using the conditions created during after the Weissenberg effect, and combinations thereof. Furthermore, in some embodiments, the back-flow material may be recycled or may be reabsorbed by surrounding tissue following clearing of sampling unit and filter.

At block 208, the wearable or insertable device detects and/or measures at least one biomarker. In some embodiments, the wearable device contains a detection module that detects the presence of targeted biomolecules, in order to determine the presence or absence of the targeted biomolecule. In other embodiments, where a quantitative measurement may be desirable, an assay module may perform a biochemical assay on the sample. The assay module may perform biochemical assays using chemical, electrical, optical, or other energy-based approaches, and/or any other conventional assay technique. It is to be understood that the use of a detection module and an assay module are not mutually exclusive, and in some embodiments both may be present in the wearable or insertable device.

At block 210, the wearable or insertable device, based on the results of the measurements from block 208, infers information about the physiological condition of the patient. For example, memory (not depicted) of the wearable or insertable device may be preprogrammed with a lookup table or other similar data that enables the logic to determine information regarding a physiological condition based on the measurement of the one or more biomarkers in the sample collected by the sampling unit.

In some embodiments, a wearable or insertable device configured with selected aspects of the present disclosure may be communicatively coupled with various remote computing devices in order to exchange data. For example, the coupling may include one or more wired or wireless communication interfaces that may be used to exchange data with one or more remote computing devices using various technologies, such as Bluetooth, Wi-Fi, ultra-wide band (UWB), etc. In some embodiments, this coupling allows for display (video, audio, or any other known means) of data.

While embodiments described herein are directed primarily to wearable apparatuses that patients affix to outer surfaces of their skin, this is not meant to be limiting. Various techniques and mechanisms described herein are equally applicable to devices that may be inserted beneath a patient's skin. FIG. 3 depicts an insertable apparatus 300 that has been inserted subcutaneously in the dermis 316 of a patient's tissue 307. Many of the components depicted in FIG. 1 are also depicted in FIG. 3, such as the sampling unit 103, regeneration unit 130, detection module 170, assay module 180, power unit 160, and logic 140, and therefore are numbered similarly. Unlike other embodiments described above, insertable apparatus 300 includes microneedles 306 protruding from both first side 304 and second side 305. And while not depicted in FIG. 3, in some embodiments, microneedles 306 may protrude from other surfaces of base (or substrate) 302, such as the sides (i.e., transversely to the outer surface of the patient's skin). Moreover, while base 302 and other bases depicted herein have been generally cuboid, this is not meant to be limiting. In various embodiments, base 302 and other bases described herein may have other shapes, such as cylindrical, spherical, pyramidal, or any other two or three dimensional shape or volume.

While the insertable device 300 of FIG. 3 is shown inserted into the tissue 307 intradermally, this is not meant to be limiting. In various embodiments, insertable device 300 may be inserted into other depths, depending on what sensing and/or dilating/ablating purposes it is meant to achieve. For example, in some embodiments, insertable apparatus 300 may be inserted into tissue 307 in deeper layers of tissue, e.g., into the hypodermis (a.k.a. the subcutaneous fat layer, adipose tissue) of tissue 307. It should be understood that in various embodiments, one or more features described with respect to each embodiment depicted in each figure may be incorporated, alone or in combination with other disclosed features, into any other embodiment described herein, as well into other embodiments not explicitly described herein.

The wearable or insertable device and methods described herein may be utilized for long term continuous or periodic monitoring, by providing a regeneration unit that prevents long term clogging or obstruction of the sampling unit by introducing fluid back-flow. The method of inducing back-flow may vary and the biomarkers monitored may vary depending on the diagnostic, therapeutic, and management goals of the individual patient.

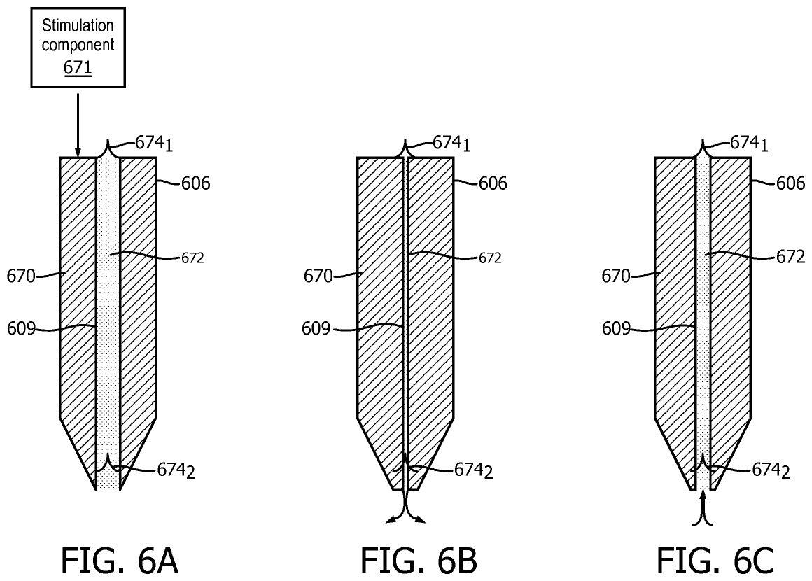

FIGS. 6A, 6B and 6C demonstrate (in cross section) another aspect of the present disclosure in which a microneedle 606 that may be employed with various embodiments described herein or by itself includes mechanically responsive material 670 that, for example defines inner lumen 609 and/or forms an inner lining of inner lumen 609. In some embodiments, mechanically-responsive material 670 may include a (e.g., continuous) deposition of materials. Mechanically-responsive material 670 may take various forms and may react mechanically to various types of stimuli. These stimuli may include one or more of thermal stimuli (e.g., changes in heat and/or heat gradients), electricity, chemical exposure, application of a magnetic field, acoustic stimuli (e.g., ultrasonic waves), and/or optical stimuli (e.g., ultraviolet light, visible light, etc.). Mechanically responsive material 670 may be stimulated (or activated) to induce various types of mechanical responses, such as expansion, contraction, predetermined movement, or any combination thereof, which in turn may purge fluid from inner lumen 609 and/or draw fluid into inner lumen 609.

In various embodiments, these stimuli may be applied by one or more stimulation components 671, such as light sources (e.g., light-emitting diodes, alone or in combination with various optical component such as collimators, light guides, lenses, etc.), piezoelectric components, speakers, chemical injectors, magnets, electrically conductive contacts, thermally-conductive contacts, and so forth. One or more stimulation components 671 may be arranged at various positions relative to microneedle 606, such as at its base, along its length, near its tip, or elsewhere in a base/substrate (e.g., 102, 302). In various embodiments, one or more stimulation components 671 may be operated to provide one or more of the aforementioned stimuli based on user input (e.g., the user presses a button or speaks a command), periodically (e.g., according to a schedule), or otherwise automatically (e.g., in response to various events, such as reservoir 104 being filled or emptied, or failing to fill or empty). For the sakes of brevity and clarity, only a single stimulation component 671 is depicted in FIG. 6A, but may be present elsewhere. In some embodiments in which a plurality of stimulation components 671 are provided, subsets of the plurality of stimulation components 671 may be selectively operated to provide stimuli to subsets of a plurality of microneedles, e.g., so that fluid is purged from some microneedles while fluid is drawn into other microneedles.

In some embodiments, mechanically-responsive material 670 may take the form of an electroactive polymer (“EAP”) that reacts mechanically, for instance, to electricity. Additionally or alternatively, in various embodiments, mechanically-responsive material 670 may take the form of magnetorheological elastomer (“MRE”) that reacts mechanically, for instance, to application of a magnetic field. MRE's may be a class of solids that include a polymeric matrix with embedded micro- or nano-sized ferromagnetic particles. In some embodiments, these particles may include carbonyl iron. Additionally or alternatively, in various embodiments, mechanically-responsive material 670 may take the form of shape-memory material such as shape-memory polymer that reacts mechanically, for instance, to a change of temperature. Additionally or alternatively, in various embodiments, mechanically-responsive material 670 may take the form of light-activated liquid crystal networks that react mechanically, for instance, to various forms of light (electromagnetic radiation).

In some embodiments, mechanically-responsive material 670 may include material (e.g., a coating) that is transitionable between a hydrophilic state in which it attracts fluid, and a hydrophobic state in which it repels fluid, similar to the embodiment depicted in FIG. 5. In some such embodiments, this material may be so transitioned using techniques such as the electrowetting electrodes 501₁₋ₙ depicted in FIG. 5. In some embodiments, a (N-dodecyltrimethoxysilane)-modified three-dimensional copper foam may be employed that can be transitioned between a hydrophilic state and a hydrophobic state using electrode processes such as those described in relation to FIG. 5. Additionally or alternatively, in some embodiments, amorphous fluoropolymers may be employed and may be transitioned between hydrophilic and hydrophobic states using, for example, applied voltage. Additionally or alternatively, in some embodiments, a material having molecules with a hydrophobic part that can be altered (e.g., inward) in response to electromagnetic radiation (e.g., ultraviolet or visible light) may be employed.

In FIG. 6A, mechanically-responsive material 670 is fully contracted so that inner lumen 609 is at its widest diameter. In FIG. 6B, some stimulus (e.g., heat, electricity, light, magnetic field, acoustical waves, etc.) has been applied to mechanically-responsive material 670 to induce a first mechanical response in the form of an expansion. Consequently, inner lumen 609 has been shrunk to a relatively small diameter (which in some embodiments may be entirely closed). This shrinking in turn causes fluid 672 (e.g., blood, interstitial fluid, etc.) within inner lumen 609 to be purged from inner lumen 609 into surrounding tissue (not depicted) as indicated by the arrows at bottom. This purging may serve to, for instance, clean inner lumen 609. Additionally or alternatively, this expansion of mechanically-responsive material 670 may also purge fluid 672 back into one or more reservoirs (e.g., 104) of a base or substrate (e.g., 102, 302), e.g., so that samples from the newly-captured fluid may be analyzed. By contrast, in FIG. 6C, the stimulus is no longer applied (or a different stimulus is applied) to induce a second mechanical response in mechanically-responsive material 670. In particular, mechanically-responsive material 670 is now contracting, which draws fluid 672 into inner lumen 609 from the surrounding tissue as indicated by the arrows at bottom. Additionally or alternatively, the contraction of FIG. 6C may draw fluid from one or more reservoirs 104 into inner lumen 609.

In FIGS. 6A, 6B and 6C, a stimulus was applied to induce mechanical expansion of mechanically-responsive material 670, and the stimulus was withdrawn to induce mechanical contraction of mechanically-responsive material 670. However, this is not meant to be limiting. In some embodiments, a stimulus may be applied to cause contraction, and the stimulus may be withdrawn (or different stimulus applied) to cause expansion. Moreover, in some embodiments, one or more valves, such as a base valve 674₁ and/or a distal valve 674₂, may be employed at or near a microneedle base and/or tip, respectively. The state(s) of these valves 674 (e.g., open or closed) when mechanically-responsive material 670 is activated may dictate which direction fluid 672 flows, into surrounding tissue or into a reservoir. In some embodiments, base valve 674₁ may be closed, for instance, while mechanically-responsive material 670 contracts, e.g., to prevent backflow from a reservoir (e.g., 104) into inner lumen 609. When inner lumen 609 is filled with fluid and base valve 674₁ is open, fluid may be drawn into a reservoir using other passive or active fluid transportation mechanisms, such as capillary forces.

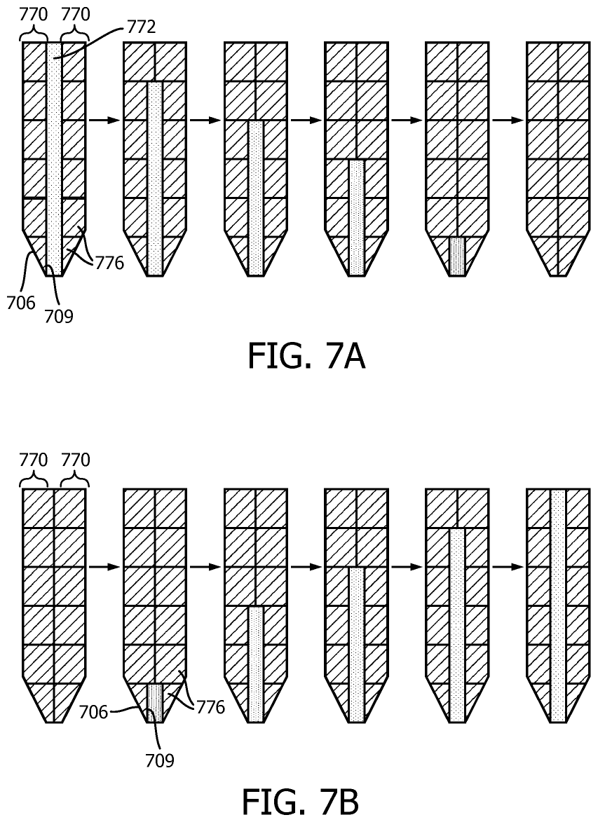

FIGS. 7A and 7B depict (in cross section) an alternative embodiment similar to that depicted in FIGS. 6A, 6B and 6C, except that mechanically-responsive material 770 is divided into a plurality of segments 776 (only two of which are indicated for the sakes of brevity and clarity) that may be individually controllable, e.g., by selectively applying one or more of the aforementioned stimuli to the segments 776 individually. By controlling the timing with which the different segments 776 are stimulated to expand and/or contract, the direction of the induced flow through inner lumen 709 can be finely controlled. In some embodiments, employing a plurality of individually-controllable segments 776 may obviate the need for one or more valves (e.g., 674 in FIGS. 6A, 6B and 6C), although their use is not foreclosed, either.

In FIG. 7A, the same microneedle 706 is depicted at various stages of operation, as indicated by the arrows. At far left, all segments 776 are contracted so that inner lumen 709 is at its widest diameter. In the second from left image, a first mechanical response in the form of expansion has been induced in two opposing segments 776 (or a single ring-shaped element) near the base of microneedle 706. This begins the process of flushing fluid from inner lumen 709. Moving to the right, in each image, more and more segments 776 are expanded in a similar manner, e.g., sequentially along a longitudinal axis of microneedle in a direction from its base to tip. Consequently, at far right, all fluid has been purged from inner lumen 709 into the surrounding tissue (not depicted).

FIG. 7B depicts the opposite of FIG. 7A. At far left in FIG. 7B, all segments 776 remain expanded. In the second image from left, the distal-most segments 776 have been contracted, beginning the process of drawing fluid into inner lumen 709. Moving to the right, in each image of FIG. 7B, more and more segments 776 are contracted in a similar manner, e.g., sequentially along a longitudinal axis of microneedle in a direction from its tip to base. Consequently, at far right, inner lumen 709 is full of fluid, which may then be drawn into a reservoir (not depicted) using, for instance, capillary forces.

The sequences of expansions/contractions depicted in FIGS. 7A and 7B are not meant to be limiting. Segments 776 may be expanded/contracted in various orders and/or at various times relative to other segments in order to draw fluid into, or purge fluid from, inner lumen 709. In some embodiments, a stimuli may be applied at one extreme end of microneedle 706 or the other (i.e. at the base or at the tip) such that as the stimuli increases (e.g., temperature increases, voltage increases, etc.), segments begin to expand (or contract) in sequence. For example, in some embodiments, an elongate thermally conductive material such as metal or copper may be placed within or near the segments 776 along the longitudinal axis of microneedle 706, and heat may be applied at one end (e.g., at the base or tip of microneedle 706). As the elongate thermally conductive material heats from one end to the other, the segments may expand or contract accordingly. Of course, other types of stimuli may be used instead.

In some embodiments, after the filling phase (FIG. 7B), the segment 776 closest to the microneedle tip may be expanded, followed by the next segment 776 in a stepwise approach—in this case from tip to base. This process pushes the fresh analysis fluid into, for instance, a reservoir (e.g., 104). In some embodiments, such device-feeding process can be recursively concatenated with the filling process (depicted in FIG. 7B), thus creating a constant flow of analysis fluid into and throughout the device. This may eliminate the need for using other means of fluid transportation inside microneedle 706, although other means may nonetheless be employed in conjunction with expansion/contraction of segments 776.

In some embodiments, microneedle (e.g., 106, 306, 406, 506, 606, 706) may be constructed so that there is a gradual change in the dimensions of the microneedle and/or the thickness of the mechanically-responsive material. When there is a thickness gradient over the microneedle's length—and the mechanically-responsive material expands—the fluid may be purged from of the inner lumen, either towards surrounding tissue or into a reservoir, depending on the gradient direction.

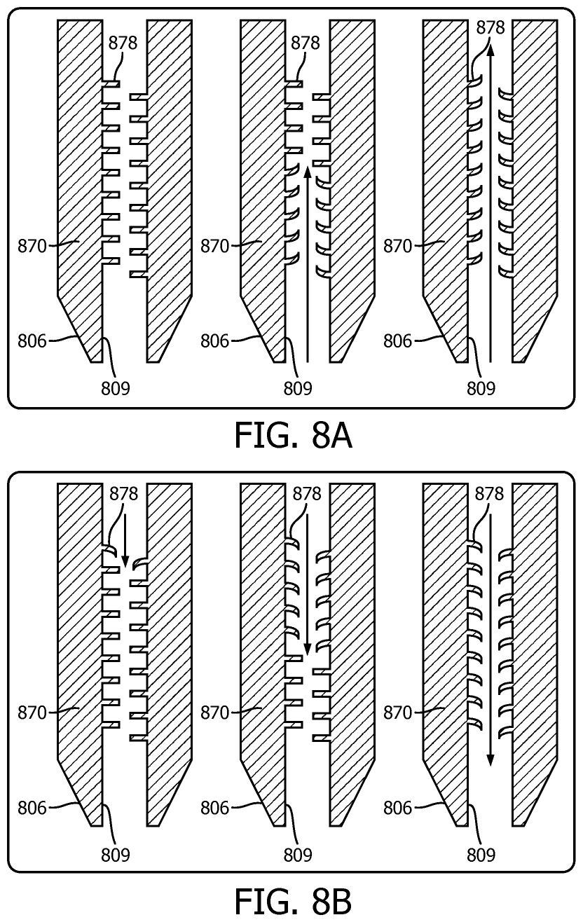

FIGS. 8A and 8B depict an alternative embodiment in which a microneedle 806 that includes an inner lumen 809 is equipped with mechanically-responsive material 870 that defines one or more paddles 878 (only one of which is designated for the sake of clarity) that extend from an inner surface of inner lumen 809 into inner lumen 809. In various embodiments, one or more paddles 878 may be operable to purge fluid from, or draw fluid into, inner lumen 809. For example, in FIG. 8A, a plurality of individually-operable paddles 878 may be operated (e.g., induced to mechanically react) in a predetermined sequence to draw fluid into inner lumen 809. At far left in FIG. 8A, no paddles 878 are yet operated. In the middle portion of FIG. 8A, the bottom paddles 878 have been induced to react mechanically to swing upwards (i.e., towards the base of microneedle 806) to initiate a flow, as indicated by the upward arrow. At far right, all paddles 878 have been induced to react mechanically, increasing the flow. In other embodiments, paddles 878 may be operated in a different order, such as in reverse.

In FIG. 8B, the plurality of individually-operably paddles 878 are operated (e.g., induced to mechanically react) in a predetermined sequence to purge fluid from inner lumen 809. At far left in FIG. 8B, only the two paddles 878 closest to the base of microneedle 806 have been activated, initiating a flow of fluid from inner lumen 809. Moving to the right, more and more paddles 878 are activated in a sequence from the base of microneedle 806 to its tip, increasing the outward flow.

In various embodiments, paddles 878 may extend completely around the inner surface that defines inner lumen 809, such that each paddle would appear as a ring if removed. Additionally or alternatively, in some embodiments, each paddle may extend less than completely around the inner surface that defines inner lumen 878, and each paddle 878 may have various shapes, such as an oar shape, a polygon, etc. In some embodiments, a cyclic motion may be established amongst paddles 878, e.g., between paddles 878 at opposite positions along the longitudinal axis of microneedle 806, to create a net drag around the paddles 878 in one direction or another. In some embodiments, only the paddles 878 may be constructed with mechanically-responsive material 870, and the paddles 878 may be secured to an inner surface of lumen 809 that is constructed with different, e.g., non-mechanically-responsive material, such as thermally conductive material in which a heat gradient may be induced.

In FIGS. 8A and 8B, paddles 878 on opposing sides of inner lumen 809 are offset from each other in a direction parallel to a longitudinal axis of microneedle 806 and do not extend more than halfway across inner lumen 809. However, this is not meant to be limiting. In some embodiments, pairs of paddles 878 may be positioned directly across inner lumen 809 from one another, and/or may extend at least halfway across inner lumen 809. In some such embodiments, opposing paddles may be actuated simultaneously to operate as a valve that can be opened and closed.

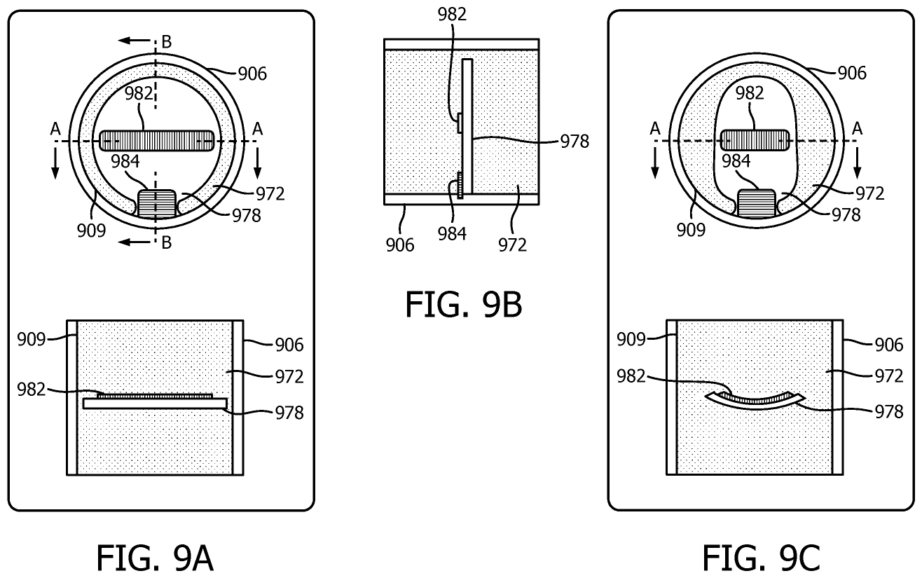

In some embodiments, a flexible substrate may be added to a paddle such as paddles 878 in FIGS. 8A and 8B to enable conversion of lateral expansion of the mechanically-responsive material into a configurable bending motion. Such a technique may provide reasonable compromise between stroke, force and actuation speed. One example of how this may be accomplished is depicted in FIGS. 9A, 9B and 9C. FIGS. 9A and 9B relate to a first state and FIG. 9C relates to a second state. FIGS. 9A and 9C each depict two views, a top down view into a lumen of a microneedle 906 and a cross-sectional view of the microneedle 906 from the line labeled “A”. FIG. 9B depicts a side cross-sectional view of microneedle 906 from the line labeled “B” and is depicted in the first state of FIG. 9A. In the top image of FIG. 9A, a paddle 978 is connected at one end to an interior wall of microneedle 906 that defines inner lumen 909. Paddle 978 includes a folding actuator 982 near its center and a bending actuator 984 near where paddle 978 connects to the wall of inner lumen 909. Fluid is indicated at 972.

In some embodiments, bending actuator 984 may be constructed at least in part with one or more of the aforementioned mechanically-responsive materials. Consequently, bending actuator 984 may be operable (e.g., mechanically induced) to bend paddle 978 up or down (e.g., upstream/downstream) within inner lumen 909, as was depicted in FIGS. 8A-B. The consequent bending actuation may be used, for example, for pumping at low actuation. As noted above, in some embodiments in which pairs of paddles 978 oppose each other across inner lumen 909, the bending actuation may be used to cause pairs of paddles 978 to act as a valve to seal and open inner lumen 909, e.g., at high actuation.

Folding actuator 982 may be constructed with a mechanically responsive material that, when exposed to the various stimuli described herein, folds upon itself, which consequently causes a blade portion of the paddle 978 to fold. This folding is best seen at bottom of FIG. 9C, in which both folding actuator 982 and, consequently, paddle 978, are folded into a U-shape. In some embodiments, paddle 978 may be kept folded (as depicted in FIG. 9C) while in the retraction phase paddle 978 to allow fluid 972 to flow around it. Once paddle 978 is in a position to make a next stroke paddle 978 may unfold (as depicted in FIG. 9A) so it takes fluid along in the next pumping cycle.

As noted above, in some embodiments, mechanically-responsive material may be constructed at least in part with activate-able liquid crystal networks, such as light-activated liquid crystal networks. Light-switchable surface topographies such as light-activated liquid crystal networks can be used to create various types of peristaltic fluid movement and/or, instead of merely expanding or contracting, may be used to create desired fluid channels to control fluid flow and/or fluid flow rates. When light-activated liquid crystal networks are suitably arranged and correctly designed, they can be selectively activated to, for instance, create fluid flow channels that modify the fluid flow inside the microneedle. Additionally or alternatively, such surfaces could be designed and used to move fluid faster through the needles as volume can be periodically displaced by switching on/off the topography.

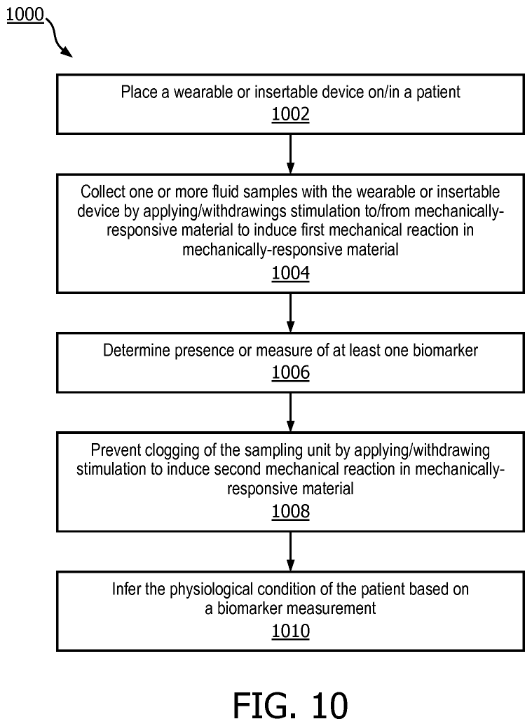

FIG. 10 depicts an example method 1000 for practicing selected aspects of the present disclosure, in accordance with various embodiments. While operations of method 1000 are shown in a particular order, this is not meant to be limiting. One or more operations may be reordered, omitted or added. At block 1002, a wearable or insertable device (e.g., 100, 300) may be placed on a patient (e.g., as a patch or e-tattoo) or inside of tissue of a patient.

At block 1004, one or more fluid samples may be collected with the wearable or insertable device. In various embodiments, this collection may include applying stimulation to, or withdrawing stimulation from, mechanically responsive material (e.g., 670, 770, 870) within an inner lumen of one or more microneedles (e.g., 106, 306, 406, 506, 606, 706, 806, 906) of the wearable or insertable device to induce a first mechanical response (e.g., contraction, swinging of paddles 878, creation of microchannels) in the mechanically-responsive material.

At block 1006, a presence or measure of at least one biomarker may be determined from the collected one or more fluid samples, e.g., by detection module 170 and/or assay module 180 in FIGS. 1 and 3. At block 1008, clogging of the one or more microneedles may be prevented by applying stimulation to, or withdrawing stimulation from, the mechanically responsive material to induce a second mechanical response (e.g., expansion, swinging of paddles 878, closing of microchannels, etc.) in the mechanically-responsive material. At block 1010, similar to block 210, the physiological condition may be inferred based on the presence or measurement of the at least one biomarker. In some embodiments, output indicative of the inference may be provided at one or more output components, such as an onboard acoustic device (e.g., to provide a beep), a display of a smart watch that is configured with selected aspects of the present disclosure, a wireless communication interface (e.g., to be transmitted to a remote computing device of the patient and/or of a caregiver), and so forth.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, (bio)materials, enzymes, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Although described separately, it is to be understood that any of the embodiments described herein may be used alone or in combination with any other embodiment(s) described herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and” consisting essentially of shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A wearable or insertable medical device comprising:
a base defining at least one reservoir;
at least one microneedle extending from the base, wherein the at least one microneedle is insertable into tissue and defines an inner lumen that fluidly couples the at least one reservoir with the tissue; and
a mechanically responsive material disposed on an inner surface of the at least one microneedle, wherein the inner surface of the at least one microneedle defines the inner lumen of the at least one microneedle, and the mechanically responsive material is reactive to a stimulus to undergo one or more mechanical responses.

2. The medical device of claim 1, further comprising a stimulation component that is selectively activated to provide the stimulus to the mechanically responsive material.

3. The medical device of claim 1, wherein at least one mechanical response of the one or more mechanical responses of the mechanically responsive material purges fluid from the inner lumen of the at least one microneedle.

4. The medical device of claim 3, further comprising a valve positioned between the mechanically responsive material and the at least one reservoir.

5. The medical device of claim 4, wherein the valve is closable such that the at least one mechanical response of the mechanically responsive material purges fluid from the inner lumen into the tissue.

6. The medical device of claim 4, wherein the valve is openable such that the at least one mechanical response of the mechanically responsive material purges fluid from the inner lumen into the at least one reservoir.

7. The medical device of claim 1, wherein at least one of the one or more mechanical responses of the mechanically responsive material draws fluid into the inner lumen of the at least one microneedle.

8. The medical device of claim 1, wherein a first mechanical response of the one or more mechanical responses comprises expansion of the mechanically-responsive material and a second mechanical response of the one or more mechanical responses comprises contraction of the mechanically-responsive material.

9. The medical device of claim 1, wherein the mechanically responsive material is divided into a plurality of individually-reactive segments that are arranged along a length of the at least one microneedle, wherein stimulation of the plurality of individually-reactive segments in a predetermined sequence causes the individually-reactive segments to expand in accordance with the predetermined sequence to purge fluid from, or draw fluid into, the inner lumen.

10. The medical device of claim 1, wherein the mechanically-responsive material comprises one or more paddles that extend from the inner surface into the inner lumen, wherein the one or more paddles are operable to purge fluid from, or draw fluid into, the inner lumen.

11. The medical device of claim 10, wherein the one or more paddles comprise a plurality of individually-operably paddles that are operably in a predetermined sequence to purge fluid from, or draw fluid into, the inner lumen.

12. The medical device of claim 10, wherein one or more of the paddles are operable as a valve to selectively open and close the inner lumen.

13. The medical device of claim 10, wherein at least one given paddle of the one or more paddles includes a folding actuator that is operable to fold the given paddle upon itself.

14. The medical device of claim 1, wherein the mechanically-responsive material is transitionable between a hydrophilic state in which the mechanically-responsive material attracts fluid, and a hydrophobic state in which the mechanically-responsive material repels fluid.

15. The medical device of claim 1, wherein the mechanically-responsive material is constructed with electroactive polymer (“EAP”) or magnetorheological elastomer (“MRE”).

16. The medical device of claim 1, wherein the mechanically-responsive material is constructed with shape-memory polymer or with light-activated liquid crystal networks.

17. The medical device of claim 1, wherein the stimulus comprises one or more of heat, electricity, electromagnetic radiation, and one or more acoustic waves.

18. The medical device of claim 1, wherein the stimulus comprises a magnetic field.

19. A method of inferring a physiological condition of a patient, comprising:
collecting one or more fluid samples with a wearable or insertable device placed on a patient, wherein the collecting comprises: applying stimulation to, or withdrawing stimulation from, mechanically responsive material within an inner lumen of one or more microneedles of the wearable or insertable device to induce a first mechanical response in the mechanically-responsive material.

20. A wearable or insertable medical device comprising:
a base defining at least one reservoir;
a plurality of microneedles extending from the base, wherein each microneedle of the plurality of microneedles is insertable into tissue and defines an inner lumen that fluidly couples the at least one reservoir with the tissue;
a mechanically responsive material deposited on inner surfaces of the plurality of microneedles, wherein the inner surfaces of the plurality of microneedles define the inner lumens of the plurality of microneedles, and the mechanically responsive material is reactive to a stimulus; and
a plurality of stimulation components that are selectively operable to provide the stimulus to mechanically responsive material of two or more subsets of the plurality of microneedles, wherein a first mechanical response of the mechanically responsive material purges fluid from the inner lumens of at least one of the two or more subsets of microneedles, and a second mechanical response of the mechanically responsive material draws fluid into the inner lumens of at least one of the two or more subsets.
